(19) United States
(12) Patent Application Publication
(10) Pub. No.: US 2024/0301210 A1
(43) Pub. Date: Sep. 12, 2024

(54) FLUORINATED PHTHALOCYANINE COMPOUND, COLORING COMPOSITION, AND INKJET INK

(71) Applicants: FUJIFILM CORPORATION, Tokyo (JP); FUJIFILM WAKO PURE CHEMICAL CORPORATION, Osaka (JP)

(72) Inventors: Yoshihiko FUJIE, Shizuoka (JP); Hirotaka SATOU, Shizuoka (JP); Sho SHIBUSAWA, Shizuoka (JP); Yoshihiro JIMBO, Shizuoka (JP); Takayuki ITO, Shizuoka (JP); Keizo KIMURA, Shizuoka (JP)

(21) Appl. No.: 18/663,097

(22) Filed: May 14, 2024

Related U.S. Application Data
(63) Continuation of application No. PCT/JP2022/041743, filed on Nov. 9, 2022.

(30) Foreign Application Priority Data
Nov. 19, 2021 (JP) 2021-188790

(51) Int. Cl. C09B 47/067; C09D 11/322

(52) U.S. Cl. CPC C09B 47/0671; C09D 11/322

(57) ABSTRACT

A fluorinated phthalocyanine compound represented by Formula (1) (in Formula (1), M represents a metal atom or an oxide of a metal atom, and R¹⁰¹ to R¹⁰⁸ each independently represent an alkyl group, an aryl group, or a heterocyclic group, provided that at least one of R¹⁰¹ to R¹⁰⁸ is a group represented by Formula (2); in Formula (2), R²⁰¹ to R²⁰⁵ each independently represent a hydrogen atom or a monovalent substituent, provided that at least one of R²⁰¹ to R²⁰⁵ is a group represented by any one selected from the group consisting of Formula (3), Formula (4), Formula (5), Formula (6), and Formula (7)); and applications thereof.

### CROSS-REFERENCE TO RELATED APPLICATION

This application is a continuation of International Application No. PCT/JP2022/041743, filed Nov. 9, 2022, which claims priority to Japanese Patent Application No. 2021-188790, filed Nov. 19, 2021. Each of the above applications is hereby expressly incorporated by reference, in its entirety, into the present application.

### BACKGROUND OF THE INVENTION

### 1. Field of the Invention

The present disclosure relates to a fluorinated phthalocyanine compound, a coloring composition, and an inkjet ink.

### 2. Description of the Related Art

A fluorinated phthalocyanine compound has been widely used, for example, as a colorant. As the colorant, for example, it is required to have a desired color tone, and to have good fastness typified by light fastness.

For example, JP1991-345861A (JP-H5-345861A) discloses a fluorine-containing phthalocyanine compound in which a fluorine atom is directly bonded to a phthalocyanine nucleus. In addition, JP2006-342264A discloses a phthalocyanine compound having a polymerizable substituent at a molecular terminal.

### SUMMARY OF THE INVENTION

An object of one embodiment of the present disclosure is to provide a fluorinated phthalocyanine compound exhibiting green color and having excellent light fastness.

An object of another embodiment of the present disclosure is to provide a coloring composition or an inkjet ink, containing the fluorinated phthalocyanine compound.

The present disclosure includes the following aspects.

<1> A fluorinated phthalocyanine compound represented by Formula (1).

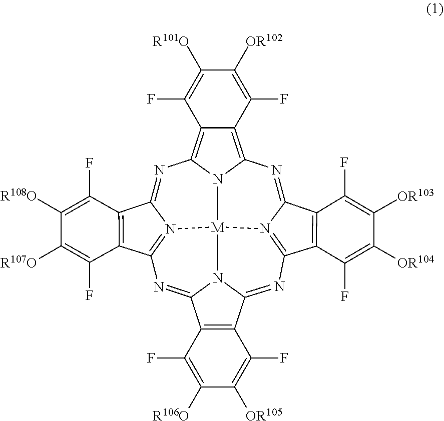

In Formula (1), M represents a metal atom or an oxide of a metal atom, and R¹⁰¹, R¹⁰², R¹⁰³, R¹⁰⁴, R¹⁰⁵, R¹⁰⁶, R¹⁰⁷, and R¹⁰⁸ each independently represent a substituted or unsubstituted alkyl group, a substituted or unsubstituted aryl group, or a substituted or unsubstituted heterocyclic group. Here, at least one of R¹⁰¹, R¹⁰², R¹⁰³, R¹⁰⁴, R¹⁰⁵, R¹⁰⁶, R¹⁰⁷, or R¹⁰⁸ is a group represented by Formula (2).

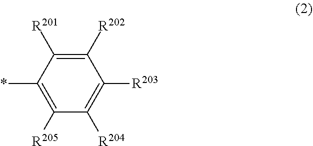

In Formula (2), R²⁰¹, R²⁰², R²⁰³, R²⁰⁴, and R²⁰⁵ each independently represent a hydrogen atom or a monovalent substituent. Here, at least one of R²⁰¹, R²⁰², R²⁰³, R²⁰⁴, or R²⁰⁵ is a group represented by any one selected from the group consisting of Formula (3), Formula (4), Formula (5), Formula (6), and Formula (7).

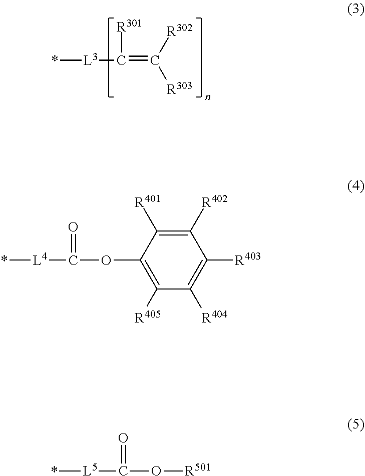

*-L⁶-OH (6)

*-L⁷-NH₂ (7)

In Formula (3), R³⁰¹, R³⁰², and R³⁰³ each independently represent a hydrogen atom or a monovalent substituent, L³ represents a single bond, a divalent linking group, or a trivalent linking group, and n represents 1 or 2.

In Formula (4), R⁴⁰¹, R⁴⁰², R⁴⁰³, R⁴⁰⁴, and R⁴⁰⁵ each independently represent a hydrogen atom or a monovalent substituent, and L⁴ represents a single bond or a divalent linking group.

In Formula (5), L⁵ represents a single bond or a divalent linking group, R⁵⁰¹ represents a hydrogen atom, a fluoroalkyl group, —CR⁵⁰²R⁵⁰³R⁵⁰⁴, or —CHR⁵⁰⁵OR⁵⁰⁶, R⁵⁰², R⁵⁰³, R⁵⁰⁴, R⁵⁰⁵, and R⁵⁰⁶ each independently represent a substituted or unsubstituted alkyl group, and R⁵⁰⁵ and R⁵⁰⁶ may be bonded to each other to form a ring.

In Formula (6), L⁶ represents a single bond or a divalent linking group.

In Formula (7), L⁷ represents a single bond or a divalent linking group.

<2> The fluorinated phthalocyanine compound according to <1>,
in which each of R¹⁰¹, R¹⁰², R¹⁰³, R¹⁰⁴, R¹⁰⁵, R¹⁰⁶, R¹⁰⁷, and R¹⁰⁸ is the group represented by Formula (2).

<3> The fluorinated phthalocyanine compound according to <1> or <2>,
in which, in Formula (2), any one of R²⁰², R²⁰³, or R²⁰⁴ is a group represented by any one selected from the group consisting of Formula (3), Formula (4), Formula (5), Formula (6), and Formula (7), and the remaining two of R²⁰², R²⁰³, and R²⁰⁴, R²⁰¹, and R²⁰⁵ are all hydrogen atoms.

<4> The fluorinated phthalocyanine compound according to any one of <1> to <3>,
in which, in Formula (2), R²⁰³ is a group represented by any one selected from the group consisting of Formula (3), Formula (4), Formula (5), Formula (6), and Formula (7), and R²⁰¹, R²⁰², R²⁰⁴, and R²⁰⁵ are all hydrogen atoms.

<5> The fluorinated phthalocyanine compound according to any one of <1> to <4>,
in which M is copper, zinc, or oxyvanadium.

<6> The fluorinated phthalocyanine compound according to any one of <1> to <5>,
in which M is copper or zinc.

<7> The fluorinated phthalocyanine compound according to any one of <1> to <6>,
in which M is zinc.

<8> A coloring composition comprising:
the fluorinated phthalocyanine compound according to any one of <1> to <7>.

<9> An inkjet ink comprising:
the fluorinated phthalocyanine compound according to any one of <1> to <7>.

According to one embodiment of the present disclosure, there is provided a fluorinated phthalocyanine compound exhibiting green color and having excellent light fastness.

According to another embodiment of the present disclosure, there is provided a coloring composition or an inkjet ink, containing the fluorinated phthalocyanine compound.

### BRIEF DESCRIPTION OF THE DRAWINGS

FIG. 1 is an absorption spectrum of a compound (G-1) in a diluted solution of ethyl acetate.

FIG. 2 is an absorption spectrum of a compound (G-5) in a diluted solution of ethyl acetate.

FIG. 3 is an absorption spectrum of a compound (G-16) in a diluted solution of ethyl acetate.

FIG. 4 is a 1H-NMR spectrum of the compound (G-1) in deuterated chloroform.

FIG. 5 is a 1H-NMR spectrum of the compound (G-5) in deuterated chloroform.

### DESCRIPTION OF THE PREFERRED EMBODIMENTS

Hereinafter, embodiments of the present disclosure will be described in detail. The present disclosure is not limited to the following embodiments. The following embodiments may be modified as appropriate within the scope of the purposes of the present disclosure.

In the present disclosure, a numerical range expressed using “to” means a range including numerical values before and after “to” as a lower limit value and an upper limit value. In a numerical range described in a stepwise manner in the present disclosure, an upper limit or a lower limit described in a certain numerical range may be replaced with an upper limit or a lower limit in another numerical range described in a stepwise manner. In addition, in a numerical range described in the present disclosure, an upper limit or a lower limit described in a certain numerical range may be replaced with a value described in Examples.

In the present disclosure, upon referring to an amount of each component in a composition, the amount means a total amount of a plurality of components present in the composition unless otherwise specified, in a case where a plurality of substances corresponding to individual components are present in the composition.

In the present disclosure, a combination of two or more preferred aspects or forms is a more preferred aspect or form.

Regarding description of a group (atomic group) in the present disclosure, a description in which substitution and unsubstitution are not specified includes a group having no substituent and a group having a substituent. For example, “hydrocarbon group” includes not only a hydrocarbon group having no substituent but also a hydrocarbon group having a substituent.

As a result of intensive studies, the present inventors have found a novel fluorinated phthalocyanine compound which has a structure represented by Formula (1) described later, exhibits green color, and has excellent light fastness.

The reason why the fluorinated phthalocyanine compound according to the present disclosure exhibits green color and has excellent light fastness is not clear, but it is considered that a substituent having a specific structure introduced into a molecular terminal (that is, a group represented by any one selected from the group consisting of Formula (3), Formula (4), Formula (5), Formula (6), and Formula (7)), efficiently acts to dissipate light energy while appropriately relaxing the phthalocyanine in an aggregated state.

In the present disclosure, the “green color” means a hue angle (h°) of 150° to 210°. That is, the hue angle)(h° of the fluorinated phthalocyanine compound according to the present disclosure is 150° to 210°, preferably 160° to 200°.

In the present disclosure, the hue angle is based on the L*a*b* color system standardized by International Commission on Illumination (CIE) in 1976 and standardized by JIS Z 8781-5:2013. The hue angle is calculated by an expression: hue angle (h°)=tan-1(a*/b*).

The hue angle (h°) in the present disclosure is obtained by substituting a* and b* measured by a method described in Examples later into the above expression.

### <Fluorinated Phthalocyanine Compound>

The fluorinated phthalocyanine compound according to the present disclosure is a compound represented by Formula (1).

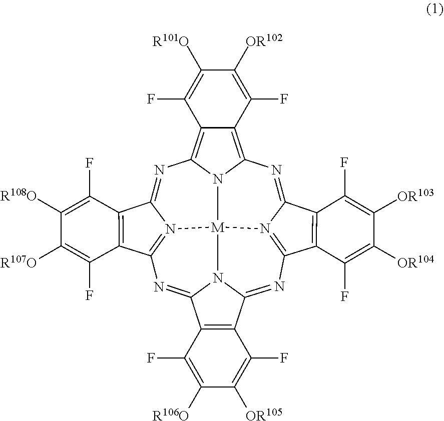

In Formula (1), M represents a metal atom or an oxide of a metal atom, and R¹⁰¹, R¹⁰², R¹⁰³, R¹⁰⁴, R¹⁰⁵, R¹⁰⁶, R¹⁰⁷, and R¹⁰⁸ each independently represent a substituted or unsubstituted alkyl group, a substituted or unsubstituted aryl group, or a substituted or unsubstituted heterocyclic group. Here, at least one of R¹⁰¹, R¹⁰², R¹⁰³, R¹⁰⁴, R¹⁰⁵, R¹⁰⁶, R¹⁰⁷, or R¹⁰⁸ is a group represented by Formula (2).

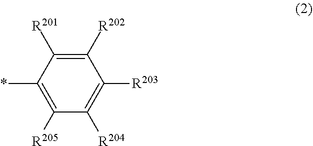

In Formula (2), R²⁰¹, R²⁰², R²⁰³, R²⁰⁴, and R²⁰⁵ each independently represent a hydrogen atom or a monovalent substituent. Here, at least one of R²⁰¹, R²⁰², R²⁰³, R²⁰⁴, or R²⁰⁵ is a group represented by any one selected from the group consisting of Formula (3), Formula (4), Formula (5), Formula (6), and Formula (7). Hereinafter, the group selected from the group consisting of Formula (3), Formula (4), Formula (5), Formula (6), and Formula (7) is also referred to as “specific substituent”.

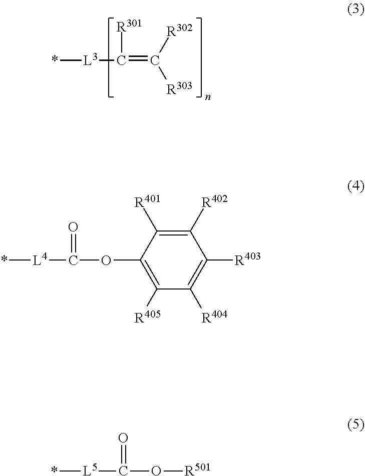

*-L¹⁶-OH (6)

*-L⁷-NH₂ (7)

In Formula (3), R³⁰¹, R³⁰², and R³⁰³ each independently represent a hydrogen atom or a monovalent substituent, L³ represents a single bond, a divalent linking group, or a trivalent linking group, and n represents 1 or 2.

In Formula (4), R⁴⁰¹, R⁴⁰², R⁴⁰³, R⁴⁰⁴, and R⁴⁰⁵ each independently represent a hydrogen atom or a monovalent substituent, and L⁴ represents a single bond or a divalent linking group.

In Formula (5), L⁵ represents a single bond or a divalent linking group, R⁵⁰¹ represents a hydrogen atom, a fluoroalkyl group, —CR⁵⁰²R⁵⁰³R⁵⁰⁴, or —CHR⁵⁰⁵OR⁵⁰⁶R⁵⁰², R⁵⁰³, R⁵⁰⁴, R⁵⁰⁵ and R⁵⁰⁶ each independently represent a substituted or unsubstituted alkyl group, and R⁵⁰⁵ and R⁵⁰⁶ may be bonded to each other to form a ring.

In Formula (6), L⁶ represents a single bond or a divalent linking group.

In Formula (7), L⁷ represents a single bond or a divalent linking group.

Hereinafter, the structure of the fluorinated phthalocyanine compound represented by Formula (1) will be specifically described.

In Formula (1), M represents a metal atom or an oxide of a metal atom.

Examples of the above-described metal atom include iron, magnesium, nickel, cobalt, copper, palladium, zinc, vanadium, titanium, indium, and tin.

Examples of the above-described oxide of a metal atom include oxides of metal atoms exemplified as the above-described metal atom, and specific examples thereof include oxytitanium (Ti═O) and oxyvanadium (V═O).

M is preferably copper, zinc, cobalt, nickel, iron, oxytitanium, or oxyvanadium, and more preferably copper, zinc, or oxyvanadium. In particular, from the viewpoint of improving the light fastness, M is preferably zinc or copper. In addition, from the viewpoint of exhibiting the green color with a hue angle of 150° to 210°, the viewpoint of case of obtaining raw materials for synthesizing the fluorinated phthalocyanine compound represented by Formula (1), the viewpoint of solubility in a solvent in a case of being applied to ink, and the like, M is preferably zinc.

In Formula (1), R¹⁰¹, R¹⁰², R¹⁰³, R¹⁰⁴, R¹⁰⁵, R¹⁰⁶, R¹⁰⁷, and R¹⁰⁸ each independently represent a substituted or unsubstituted alkyl group, a substituted or unsubstituted aryl group, or a substituted or unsubstituted heterocyclic group. Here, at least one of R¹⁰¹, R¹⁰², R¹⁰³, R¹⁰⁴, R¹⁰⁵, R¹⁰⁶, R¹⁰⁷, or R¹⁰⁸ is a group represented by Formula (2).

As the above-described substituted or unsubstituted alkyl group, an alkyl group having a total number of carbon atoms of 1 to 18 is preferable, and an alkyl group having a total number of carbon atoms of 1 to 12 is more preferable. A substituent included in the substituted alkyl group is not particularly limited, and examples thereof include a substituent S described later. Here, in a case where the alkyl group has a substituent including a carbon atom, the total number of carbon atoms in the alkyl group means the total number of carbon atoms including the number of carbon atoms in the substituent.

As the above-described substituted or unsubstituted aryl group, an aryl group having a total number of carbon atoms of 6 to 18 is preferable, an aryl group having a total number of carbon atoms of 6 to 14 is more preferable, and a group represented by Formula (2) is particularly preferable. A substituent included in the substituted aryl group is not particularly limited, and examples thereof include the substituent S described later. Among these, the specific substituent is preferable. Here, in a case where the aryl group has a substituent including a carbon atom, the total number of carbon atoms in the aryl group means the total number of carbon atoms including the carbon atoms of the substituent.

As the above-described substituted or unsubstituted heterocyclic group, a heterocyclic group having a total number of carbon atoms of 2 to 12 and including a nitrogen atom, an oxygen atom, a sulfur atom, or the like as a heteroatom is preferable, and a heterocyclic group having a total number of carbon atoms of 3 to 8 and including a nitrogen atom, an oxygen atom, a sulfur atom, or the like as a heteroatom is more preferable. A substituent included in the substituted heterocyclic group is not particularly limited, and examples thereof include a substituent S described later. Here, in a case where the heterocyclic group has a substituent including a carbon atom, the total number of carbon atoms in the heterocyclic group means the total number of carbon atoms including the number of carbon atoms in the substituent.

From the viewpoint of case of synthesis, the viewpoint of exhibiting the green color with a hue angle of 150° to 210°, and the viewpoint of improving the light fastness, it is preferable that, in Formula (1), four or more of at least one of R¹⁰¹ or R¹⁰², at least one of R¹⁰³ or R¹⁰⁴, at least one of R¹⁰⁵ or R¹⁰⁶, and at least one of R¹⁰⁷ or R¹⁰⁸ are the group represented by Formula (2). Among these, from the viewpoint of exhibiting the green color with a hue angle of 150° to 210° and the viewpoint of further improving the light fastness, it is preferable that each of R¹⁰¹, R¹⁰², R¹⁰³, R¹⁰⁴, R¹⁰⁵, R¹⁰⁶, R¹⁰⁷, and R¹⁰⁸ is the group represented by Formula (2).

In Formula (2), R²⁰¹, R²⁰², R²⁰³, R²⁰⁴, and R²⁰⁵ each independently represent a hydrogen atom or a monovalent substituent. Here, at least one of R²⁰¹, R²⁰², R²⁰³, R²⁰⁴, or R²⁰⁵ is a group represented by any one selected from the group consisting of Formula (3), Formula (4), Formula (5), Formula (6), and Formula (7) (that is, the specific substituent).

The above-described monovalent substituent is not particularly limited, and examples thereof include a halogen atom, an alkyl group, a cycloalkyl group, an alkenyl group, an aryl group, a heterocyclic group, a cyano group, a hydroxy group, a nitro group, an amino group, an alkylamino group, an alkoxy group, an aryloxy group, an acylamino group, an arylamino group, a ureido group, a sulfamoylamino group, an alkylthio group, an arylthio group, an alkoxycarbonylamino group, a sulfonamide group, a carbamoyl group, a sulfamoyl group, a sulfonyl group, an alkoxycarbonyl group, a heterocyclic oxy group, an azo group, an acyloxy group, a carbamoyloxy group, a silyloxy group, an aryloxycarbonyl group, an aryloxycarbonylamino group, an imide group, a heterocyclic thio group, a phosphoryl group, an acyl group, a carboxy group, and a sulfo group. Each of these groups may further have a substituent. In the present disclosure, these monovalent substituents are referred to as “substituent S”, and a monovalent substituent other than the specific substituent is referred to as “substituent T”.

The monovalent substituent is preferably the specific substituent.

In Formula (2), the number of substituents of the specific substituent is not particularly limited, but from the viewpoint of case of synthesis, the viewpoint of exhibiting the green color with a hue angle of 150° to 210°, and the viewpoint of improving the light fastness, it is preferably 1. That is, from the viewpoint of case of synthesis, the viewpoint of exhibiting the green color with a hue angle of 150° to 210°, and the viewpoint of improving the light fastness, it is preferable that any one of R²⁰¹, R²⁰², R²⁰³, R²⁰⁴, or R²⁰⁵ in Formula (2) is the specific substituent, and the remaining four of R²⁰¹, R²⁰², R²⁰³, R²⁰⁴, and R²⁰⁵ are each independently a hydrogen atom or the above-described substituent T.

In addition, from the viewpoint of exhibiting the green color with a hue angle of 150° to 210° and the viewpoint of improving the light fastness, a substitution position of the specific substituent is preferably in a meta-position or a para-position, and from the viewpoint of case of obtaining raw materials for synthesizing the fluorinated phthalocyanine compound represented by Formula (1), the substituent position of the specific substituent is more preferably in the para-position. That is, it is preferable that any one of R²⁰², R²⁰³, or R²⁰⁴ in Formula (2) is the specific substituent, the remaining two of R²⁰², R²⁰³, and R²⁰⁴, R²⁰¹, and R²⁰⁵ are each independently a hydrogen atom or the above-described substituent T; and it is more preferable that R²⁰³ is the specific substituent, and R²⁰¹, R²⁰², R²⁰⁴, and R²⁰⁵ are each independently a hydrogen atom or the above-described substituent T.

In Formula (2), from the viewpoint of exhibiting the green color with a hue angle of 150° to 210°, R²⁰¹ and R²⁰⁵ are preferably hydrogen atoms.

In addition, from the viewpoint of exhibiting the green color with a hue angle of 150° to 210° and the viewpoint of further improving the light fastness, in Formula (2), it is preferable that any one of R²⁰², R²⁰³, or R²⁰⁴ is the specific substituent, the remaining two of R²⁰², R²⁰³, and R²⁰⁴, R²⁰¹, and R²⁰⁵ are all hydrogen atoms; and it is more preferable that R²⁰³ is the specific substituent and R²⁰¹, R²⁰², R²⁰⁴, and R²⁰⁵ are all hydrogen atoms.

In Formula (1), a plurality of specific substituents may be the same or different from each other, but from the viewpoint of case of synthesis, it is preferable that a plurality of specific substituents are the same.

In addition, in Formula (1), a plurality of the groups represented by Formula (2) may be the same or different from each other, but from the viewpoint of case of synthesis, it is preferable that a plurality of the groups represented by Formula (2) are the same.

The group represented by Formula (3) has a carbon-carbon double bond at a terminal.

In Formula (3), R³⁰¹, R³⁰², and R³⁰³ each independently represent a hydrogen atom or a monovalent substituent, L³ represents a single bond, a divalent linking group, or a trivalent linking group, and n represents 1 or 2.

Examples of the above-described monovalent substituent include the above-described substituent S.

In addition, examples of the above-described divalent linking group include divalent linking groups consisting of a combination of one or two or more divalent groups of —O—, —S—, —C(═O)—, —CR^{A}R^{B}—, —C(═S)—, —NR^{C}—, —SO—, —SO₂—, a residue obtained by removing two hydrogen atoms from a hydrocarbon ring (for example, 1,4-phenylene, cyclohexane-1,4-diyl, and the like), and a residue obtained by removing two hydrogen atoms from a heterocyclic ring (for example, thiophene-2,5-diyl, pyridine-2,5-diyl, and the like). Here, R^{A}, R^{B}, and R^{C} each independently represent a hydrogen atom or a monovalent substituent. Each of these groups may further have a substituent such as the substituent S. In the present disclosure, these divalent linking groups are referred to as “linking group R”.

Here, it is preferable that R^{A}, R^{B}, and R^{C} are each independently a hydrogen atom or the substituent S, and it is more preferable that R^{A}, R^{B}, and R^{C} are each independently a hydrogen atom, an alkyl group, or the specific substituent.

Furthermore, examples of the trivalent linking group include a trivalent hydrocarbon group such as a methine group, a trivalent group of —N<, and a trivalent linking group consisting of a combination of this trivalent group and the above-described linking group R.

From the viewpoint of exhibiting the green color with a hue angle of 150° to 210° and the viewpoint of improving the light fastness, in Formula (3), it is preferable that R³⁰¹, R³⁰², and R³⁰³ are each independently a hydrogen atom or an alkyl group, and it is more preferable that all are hydrogen atoms.

From the viewpoint of exhibiting the green color with a hue angle of 150° to 210° and the viewpoint of improving the light fastness, in Formula (3), L³ is preferably a divalent or trivalent linking group, and more preferably a divalent or trivalent linking group consisting of a combination of —C(═O)— and —O—, —NH—, or —N<.

The group represented by Formula (4) has a phenoxy carbonyl group.

In Formula (4), R⁴⁰¹, R⁴⁰², R⁴⁰³, R⁴⁰⁴, and R⁴⁰⁵ each independently represent a hydrogen atom or a monovalent substituent, and L⁴ represents a single bond or a divalent linking group.

Examples of the above-described monovalent substituent include the above-described substituent S.

In addition, examples of the above-described divalent linking group include the above-described linking group R.

From the viewpoint of exhibiting the green color with a hue angle of 150° to 210° and the viewpoint of improving the light fastness, in Formula (4), it is preferable that R⁴⁰¹, R⁴⁰², R⁴⁰³R⁴⁰⁴, and R⁴⁰⁵ are all hydrogen atoms, or R⁴⁰¹, R⁴⁰², R⁴⁰⁴, and R⁴⁰⁵ are all hydrogen atoms and R⁴⁰³ is a monovalent substituent (for example, the substituent S).

In Formula (4), from the viewpoint of exhibiting the green color with a hue angle of 150° to 210° and the viewpoint of improving the light fastness, L⁴ is preferably a single bond. In Formula (5), L⁵ represents a single bond or a divalent linking group.

Examples of the above-described divalent linking group include the above-described linking group R.

In Formula (5), from the viewpoint of exhibiting the green color with a hue angle of 150° to 210° and the viewpoint of improving the light fastness, L⁵ is preferably a single bond.

In Formula (5), R⁵⁰¹ represents a hydrogen atom, a fluoroalkyl group, —CR⁵⁰²R⁵⁰³R⁵⁰⁴, or —CHR⁵⁰⁵OR⁵⁰⁶. R⁵⁰², R⁵⁰³, R⁵⁰⁴, R⁵⁰⁵, and R⁵⁰⁶ each independently represent a substituted or unsubstituted alkyl group, and R⁵⁰⁵ and R⁵⁰⁶ may be bonded to each other to form a ring. Here, the fluoroalkyl group represents an alkyl group substituted with at least one fluorine atom. Examples of a substituent included in the above-described substituted alkyl group include the substituent S.

In Formula (5), from the viewpoint of exhibiting the green color with a hue angle of 150° to 210° and the viewpoint of improving the light fastness, R⁵⁰¹ is preferably a hydrogen atom.

The group represented by Formula (6) has a hydroxy group at a terminal.

In Formula (6), L⁶ represents a single bond or a divalent linking group.

Examples of the above-described divalent linking group include the above-described linking group R.

From the viewpoint of exhibiting the green color with a hue angle of 150° to 210° and the viewpoint of improving the light fastness, in Formula (6), L⁶ is preferably a divalent linking group; more preferably a divalent linking group consisting of a combination of one or more of —C(═O)—, —O—, —CR^{A}R^{B}—, and —NR^{C}—; and still more preferably a divalent linking group consisting of a combination of one or more of —C(═O)—, —O—, —NH—, and CH₂. Here, R^{A}, R^{B}, and R^{C} each independently represent a hydrogen atom or a monovalent substituent, and preferred aspects thereof are the same as those of R^{A}, R^{B}, and R^{C} described above.

The group represented by Formula (7) has an unsubstituted amino group at a terminal.

In Formula (7), L⁷ represents a single bond or a divalent linking group.

Examples of the above-described divalent linking group include the above-described linking group R.

From the viewpoint of exhibiting the green color with a hue angle of 150° to 210° and the viewpoint of improving the light fastness, in Formula (7), L⁷ is preferably a divalent linking group; more preferably a divalent linking group consisting of a combination of one or more of —C(═O)—, —O—, —CR^{A}R^{B}—, and —NR^{C}—; and still more preferably a divalent linking group consisting of a combination of one or more of —C(═O)—, —O—, —NH—, and —CH₂—. Here, R^{A}, R^{B}, and R^{C} each independently represent a hydrogen atom or a monovalent substituent, and preferred aspects thereof are the same as those of R^{A}, R^{B}, and R^{C} described above.

From the viewpoint of solubility in a solvent in a case of being applied to ink, the specific substituent is preferably the group represented by Formula (3) or Formula (5). In addition, from the viewpoint of case of obtaining raw materials for synthesizing the fluorinated phthalocyanine compound represented by Formula (1) and the viewpoint of case of synthesis, the specific substituent is preferably the group represented by Formula (3), Formula (4), or Formula (5). Furthermore, from the viewpoint of reducing printing blurring on a printing base material such as plain paper mainly composed of cellulose and a photographic paper consisting of inorganic porous material in a case of being applied to ink, the specific substituent is preferably the group represented by Formula (6) or Formula (7).

From the viewpoint of exhibiting the green color with a hue angle of 150° to 210° and the viewpoint of improving the light fastness, the number of specific substituents in the fluorinated phthalocyanine compound represented by Formula (1) is preferably 1 to 12, more preferably 4 to 10, and still more preferably 8.

The maximal absorption wavelength of the fluorinated phthalocyanine compound according to the present disclosure is preferably in a wavelength range of 630 nm to 690 nm, more preferably in a wavelength range of 640 nm to 680 nm, and still more preferably in a wavelength range of 650 nm to 670 nm.

A molar absorption coefficient of the fluorinated phthalocyanine compound according to the present disclosure at the maximal absorption wavelength is preferably 100,000 L/(mol·cm) or more, and more preferably 120,000 L/(mol·cm) or more.

The above-described maximal absorption wavelength and molar absorption coefficient are the maximal absorption wavelength and the molar absorption coefficient in an absorption spectrum of a solution of the fluorinated phthalocyanine compound, and are measured using a spectrophotometer. A specific measuring method is as follows. That is, for example, a solution having a concentration of 1×10⁶ M is prepared using ethyl acetate, chloroform, or dimethylformamide as a solvent, and the obtained solution is measured using a spectrophotometer (for example, UV-3100 manufactured by Shimadzu Corporation) and a quartz cell with an optical path length of 10 mm.

Hereinafter, specific examples of the fluorinated phthalocyanine compound according to the present disclosure are shown as [(G-1) to (G-17)]. The fluorinated phthalocyanine compound according to the present disclosure is not limited to the following specific examples.

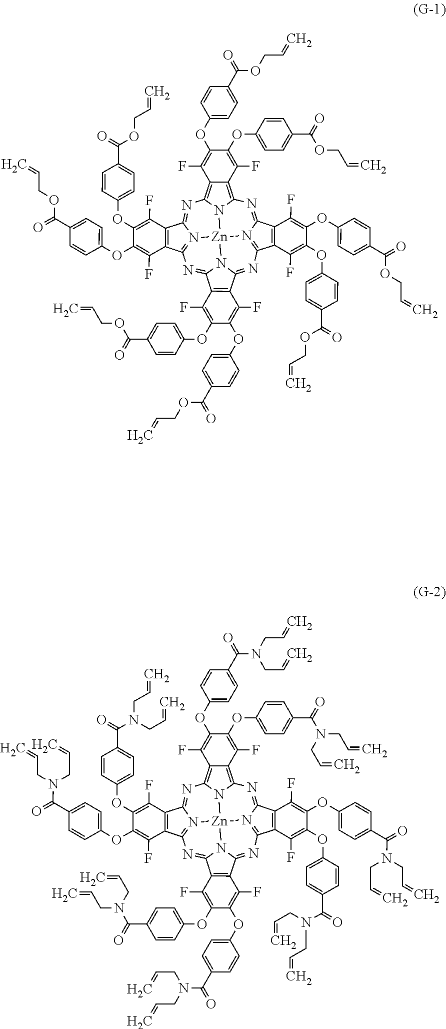

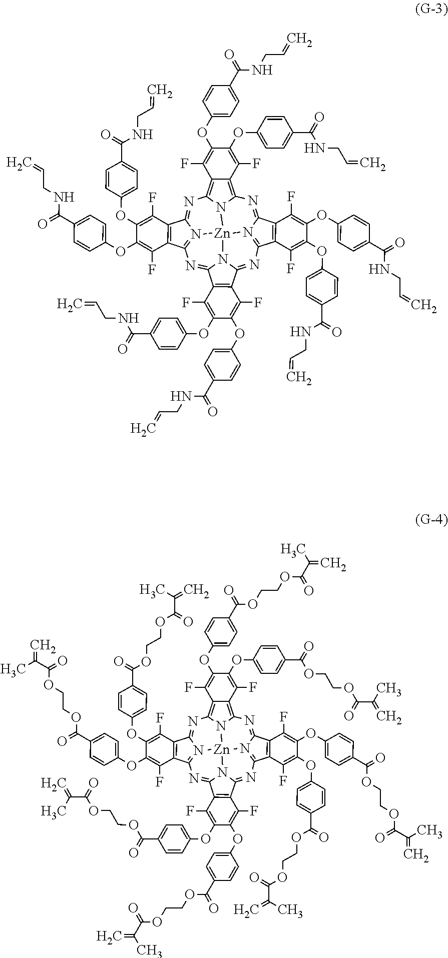

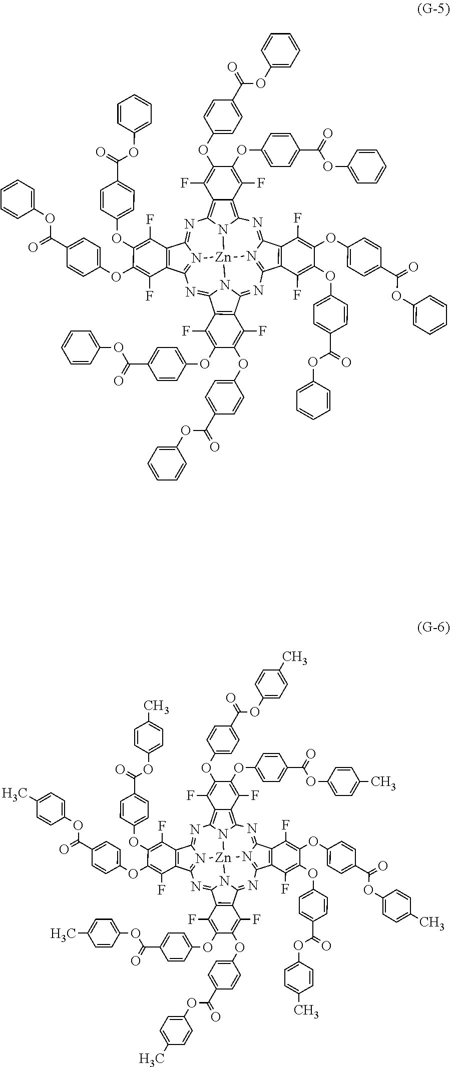

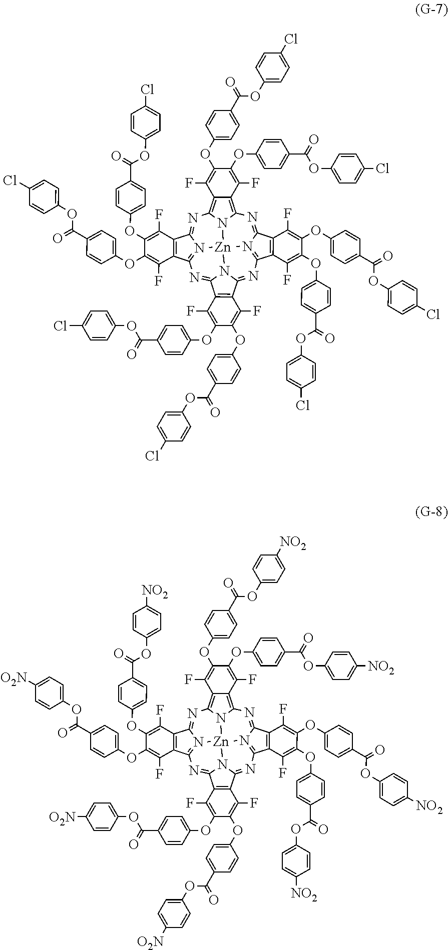

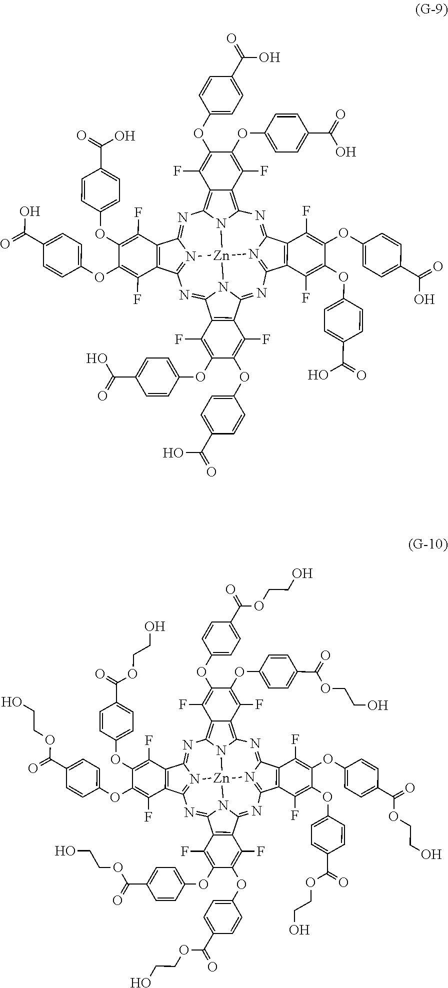

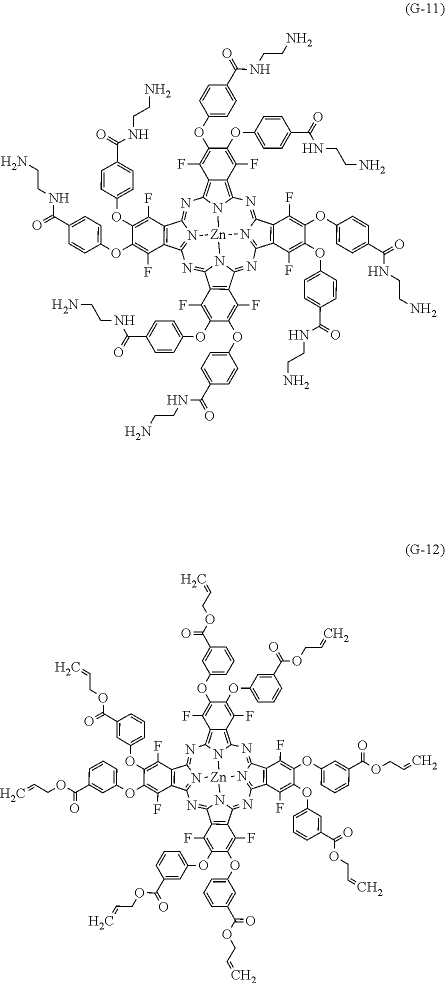

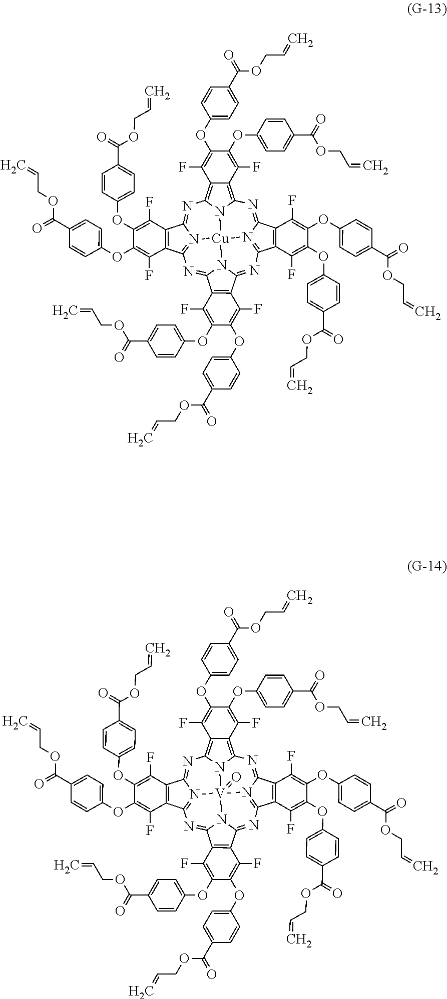

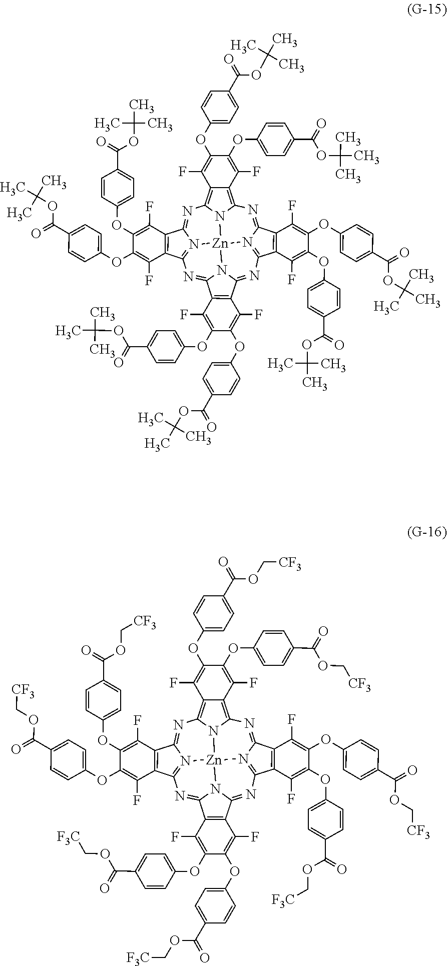

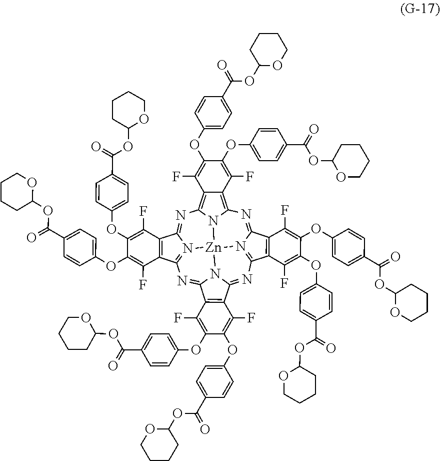

### [Synthesis Method]

A method for producing the fluorinated phthalocyanine compound according to the present disclosure is not particularly limited, and the fluorinated phthalocyanine compound may be produced, for example, by a known method or with reference to a known method. For example, the fluorinated phthalocyanine compound can be synthesized in accordance with a method described in JP2005-298491A.

Specifically, for example, 3,6-difluorophthalonitrile having various substituents at 4 and 5 positions, which can be synthesized by a known method in the related art, is reacted in a solvent such as diethylene glycol and benzonitrile at a temperature of 80° C. to 200° C. in the presence of a metal salt such as copper acetate and zinc iodide, thereby producing the fluorinated phthalocyanine compound according to the present disclosure.

### [Applications]

Since the fluorinated phthalocyanine compound according to the present disclosure exhibits green color, it can be suitably used as a colorant or a coloring agent.

In addition, since the fluorinated phthalocyanine compound according to the present disclosure exhibits green color, the fluorinated phthalocyanine compound according to the present disclosure can be suitably used in a coloring composition containing the fluorinated phthalocyanine compound according to the present disclosure or an inkjet ink containing the fluorinated phthalocyanine compound according to the present disclosure.

### <Coloring Composition>

The coloring composition according to the present disclosure is not particularly limited as long as it contains the fluorinated phthalocyanine compound according to the present disclosure.

A form of the coloring composition according to the present disclosure is not particularly limited, and may be, for example, liquid, solid, or semi-solid at 25° C.

Components other than the fluorinated phthalocyanine compound according to the present disclosure (hereinafter, also referred to as other components) contained in the coloring composition according to the present disclosure may be appropriately determined according to the application of the coloring composition.

Examples of the application of the coloring composition according to the present disclosure include various inks, paints, dyes, colored resins, color masterbatches, and colored resin pellets.

Examples of the other components contained in the coloring composition according to the present disclosure include various additives such as a solvent, a resin, a colorant other than the fluorinated phthalocyanine compound according to the present disclosure, a mold release agent, an antioxidant, a surfactant, and a preservative.

A content of the fluorinated phthalocyanine compound according to the present disclosure in the coloring composition according to the present disclosure may be appropriately determined according to the application of the coloring composition.

### <Inkjet Ink>

The inkjet ink according to the present disclosure is not particularly limited as long as it contains the fluorinated phthalocyanine compound according to the present disclosure. Examples of the inkjet ink according to the present disclosure include an ink in which the fluorinated phthalocyanine compound according to the present disclosure is dissolved and/or dispersed in a lipophilic medium or an aqueous medium.

The inkjet ink according to the present disclosure contains, in addition to the fluorinated phthalocyanine compound according to the present disclosure and the lipophilic medium or the aqueous medium, a known additive such as a colorant other than the fluorinated phthalocyanine compound according to the present disclosure, an anti-drying agent (wetting agent), an antifading agent, an emulsion stabilizer, a penetration enhancer, an ultraviolet absorber, a preservative, a fungicide, a pH adjuster, a surface tension adjuster, an antifoaming agent, a viscosity adjuster, a dispersant, a dispersion stabilizer, a rust inhibitor, and a chelating agent, as necessary.

The inkjet ink according to the present disclosure may be an ink which is cured by irradiation with active energy ray. In a case of being the ink which is cured by irradiation with active energy ray, the ink further contains, in addition to the fluorinated phthalocyanine compound according to the present disclosure and the lipophilic medium or the aqueous medium, a known additive such as a curable component (for example, a polymerizable compound, a polymerization initiator, an epoxy compound, a curing agent, and the like) and a colorant other than the fluorinated phthalocyanine compound according to the present disclosure.

A content of the fluorinated phthalocyanine compound according to the present disclosure in the inkjet ink according to the present disclosure may be appropriately determined according to the application of the ink. The content of the fluorinated phthalocyanine compound according to the present disclosure is, for example, preferably 0.5% by mass to 8% by mass and more preferably 3% by mass to 6% by mass with respect to the total mass of the inkjet ink according to the present disclosure.

### EXAMPLES

Hereinafter, the present disclosure will be described in detail according to Examples. However, the present disclosure is not limited to the following Examples.

Compounds (G-1) to (G-14) and (G-16) shown below are the same as (G-1) to (G-14) and (G-16) in the specific examples of the above-described fluorinated phthalocyanine compound according to the present disclosure, respectively.

### Synthesis Example 1: Synthesis of Compound (G-1)

388 mg of zinc iodide, 2.11 g of 3,6-difluoro-4,5-bis[4-(allyloxycarbonyl)phenoxy]phthalonitrile, and 4 mL of benzonitrile were charged into a 50 mL eggplant flask, and the mixture was reacted at 150° C. for 3 hours, 160° C. for 3 hours, and 170° C. for 2 hours under a nitrogen atmosphere. The inside of the eggplant flask was cooled to room temperature (25° C.), and crystals were re-precipitated with 60 mL of methanol, and the precipitated crystals were collected by filtration.

The obtained crystals were purified by silica gel column chromatography (developing solvent: ethyl acetate/hexane=1/1, v/v) to obtain a compound (G-1). The yielding amount was 0.44 g and the yield was 17%. Matrix-Assisted Laser Desorption Ionization (MALDI)-Time of Flight Mass Spectrometry (TOF MS): 2132 ([M+1]⁺)

The maximal absorption wavelength of an absorption spectrum of the compound (G-1) in a diluted solution of ethyl acetate was 664 nm.

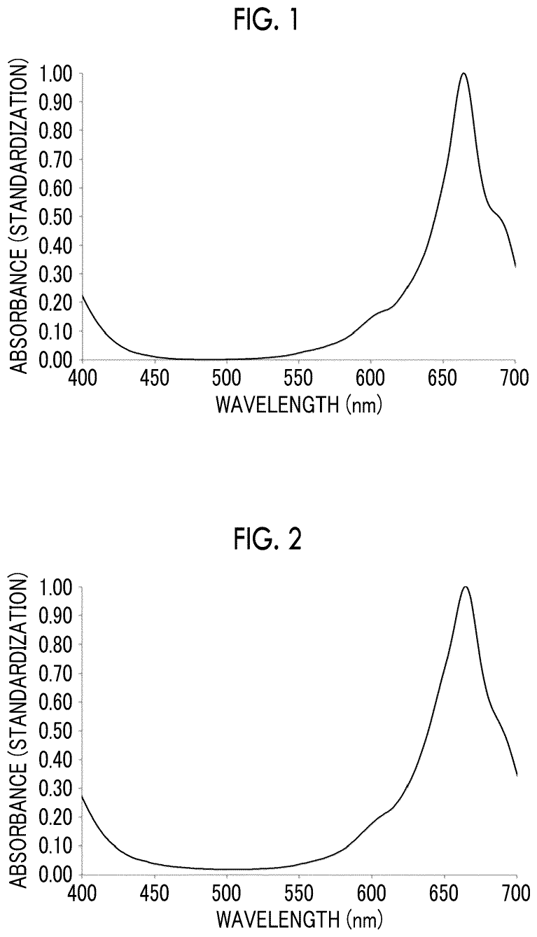

FIG. 1 shows the absorption spectrum of the compound (G-1) in the diluted solution of ethyl acetate. In addition, FIG. 4 shows a 1H-NMR spectrum of the compound (G-1) in deuterated chloroform.

### Synthesis Example 2: Synthesis of Compound (G-2)

A compound (G-2) was obtained in the same manner as in Synthesis Example 1, except that 2.11 g of 3,6-difluoro-4,5-bis[4-(allyloxycarbonyl)phenoxy]phthalonitrile was changed to 2.43 g of 3,6-difluoro-4,5-bis[4-(diallylaminocarbonyl)phenoxy)phthalonitrile. MALDI-TOF MS: 2242 ([M+1]⁺)

The maximal absorption wavelength of an absorption spectrum of the compound (G-2) in a diluted solution of ethyl acetate was 664 nm.

### Synthesis Example 3: Synthesis of Compound (G-3)

A compound (G-3) was obtained in the same manner as in Synthesis Example 1, except that 2.11 g of 3,6-difluoro-4,5-bis[4-(allyloxycarbonyl)phenoxy]phthalonitrile was changed to 2.10 g of 3,6-difluoro-4,5-bis[4-(allylaminocarbonyl)phenoxy)phthalonitrile. MALDI-TOF MS: 2122 ([M+1]⁺)

The maximal absorption wavelength of an absorption spectrum of the compound (G-3) in a diluted solution of ethyl acetate was 664 nm.

### Synthesis Example 4: Synthesis of Compound (G-4)

1.0 g of a compound (G-10) synthesized by a method described in Synthesis Example 10 later was dissolved in 10 mL of N-methylpyrrolidone, and the internal temperature was lowered to 0° C. 0.6 g of methacrylic anhydride was added thereto, and the mixture was allowed to react at room temperature for 30 minutes and at 45° C. for 60 minutes. The reaction solution was poured into water and then extracted with ethyl acetate, and the extract was purified by silica gel column chromatography (developing solvent: ethyl acetate) to obtain a compound (G-4). MALDI-TOF MS: 2705 ([M+1]⁺)

The maximal absorption wavelength of an absorption spectrum of the compound (G-4) in a diluted solution of ethyl acetate was 664 nm.

### Synthesis Example 5: Synthesis of Compound (G-5)

A compound (G-5) was obtained in the same manner as in Synthesis Example 1, except that 2.11 g of 3,6-difluoro-4,5-bis[4-(allyloxycarbonyl)phenoxy]phthalonitrile was changed to 2.40 g of 3,6-difluoro-4,5-bis[4-(phenoxycarbonyl)phenoxy)phthalonitrile. MALDI-TOF MS: 2417 ([M+1]⁺)

The maximal absorption wavelength of an absorption spectrum of the compound (G-5) in a diluted solution of ethyl acetate was 664 nm.

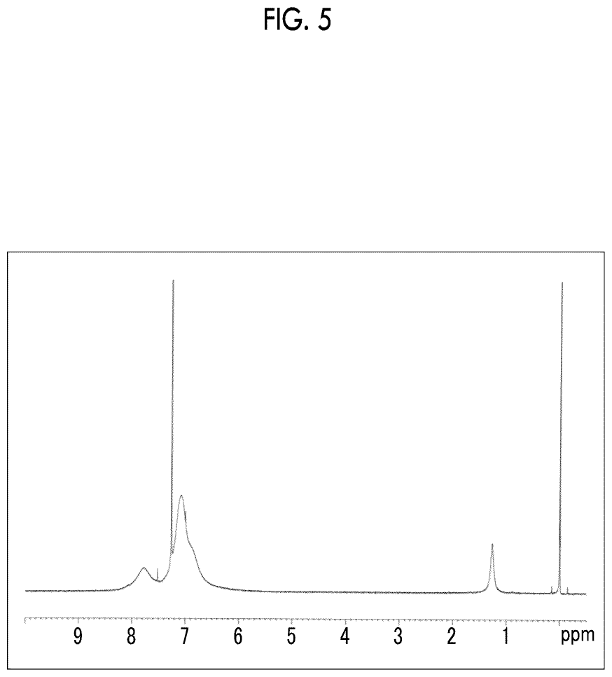

FIG. 2 shows the absorption spectrum of the compound (G-5) in the diluted solution of ethyl acetate. In addition, FIG. 5 shows a 1H-NMR spectrum of the compound (G-5) in deuterated chloroform.

### Synthesis Example 6: Synthesis of Compound (G-6)

A compound (G-6) was obtained in the same manner as in Synthesis Example 1, except that 2.11 g of 3,6-difluoro-4,5-bis[4-(allyloxycarbonyl)phenoxy]phthalonitrile was changed to 2.52 g of 3,6-difluoro-4,5-bis[4-((4-methylphenoxy))carbonyl)phenoxy)phthalonitrile. MALDI-TOF MS: 2530 ([M+1]⁺)

The maximal absorption wavelength of an absorption spectrum of the compound (G-6) in a diluted solution of ethyl acetate was 664 nm.

### Synthesis Example 7: Synthesis of Compound (G-7)

A compound (G-7) was obtained in the same manner as in Synthesis Example 1, except that 2.11 g of 3,6-difluoro-4,5-bis[4-(allyloxycarbonyl)phenoxy]phthalonitrile was changed to 3.19 g of 3,6-difluoro-4,5-bis[4-((4-chlorophenoxy)carbonyl)phenoxy)phthalonitrile. MALDI-TOF MS: 2689 ([M+1]⁺)

The maximal absorption wavelength of an absorption spectrum of the compound (G-7) in a diluted solution of ethyl acetate was 664 nm.

### Synthesis Example 8: Synthesis of Compound (G-8)

A compound (G-8) was obtained in the same manner as in Synthesis Example 1, except that 2.11 g of 3,6-difluoro-4,5-bis[4-(allyloxycarbonyl)phenoxy]phthalonitrile was changed to 2.77 g of 3,6-difluoro-4,5-bis[4-((4-nitrophenoxy))carbonyl)phenoxy)phthalonitrile. MALDI-TOF MS: 2777 ([M+1]⁺)

The maximal absorption wavelength of an absorption spectrum of the compound (G-8) in a diluted solution of ethyl acetate was 665 nm.

### Synthesis Example 9: Synthesis of Compound (G-9)

A compound (G-9) was obtained in the same manner as in Synthesis Example 1, except that 2.11 g of 3,6-difluoro-4,5-bis[4-(allyloxycarbonyl)phenoxy]phthalonitrile was changed to 1.77 g of 3,6-difluoro-4,5-bis(4-carboxyphenoxy)phthalonitrile. MALDI-TOF MS: 1809 ([M+1]⁺)

The maximal absorption wavelength of an absorption spectrum of the compound (G-9) in a diluted solution of ethyl acetate was 663 nm.

### Synthesis Example 10: Synthesis of Compound (G-10)

A compound (G-10) was obtained in the same manner as in Synthesis Example 1, except that 2.11 g of 3,6-difluoro-4,5-bis[4-(allyloxycarbonyl)phenoxy]phthalonitrile was changed to 2.14 g of 3,6-difluoro-4,5-bis[4-((2-hydroxyethyloxy))carbonyl)phenoxy)phthalonitrile. MALDI-TOF MS: 2161 ([M+1]⁺)

The maximal absorption wavelength of an absorption spectrum of the compound (G-10) in a diluted solution of ethyl acetate was 664 nm.

### Synthesis Example 11: Synthesis of Compound (G-11)

A compound (G-11) was obtained in the same manner as in Synthesis Example 1, except that 2.11 g of 3,6-difluoro-4,5-bis[4-(allyloxycarbonyl)phenoxy]phthalonitrilewas changed to 2.13 g of 3,6-difluoro-4,5-bis[4-((2-aminoethyl)oxycarbonyl)phenoxy)phthalonitrile. MALDI-TOF MS: 2153 ([M+1]⁺)

The maximal absorption wavelength of an absorption spectrum of the compound (G-11) in a diluted solution of ethyl acetate was 664 nm.

### Synthesis Example 12: Synthesis of Compound (G-12)

A compound (G-12) was obtained in the same manner as in Synthesis Example 1, except that 2.11 g of 3,6-difluoro-4,5-bis[4-(allyloxycarbonyl)phenoxy]phthalonitrilewas changed to 2.11 g of 3,6-difluoro-4,5-bis[3-(allylaminocarbonyl)phenoxy)phthalonitrile. MALDI-TOF MS: 2122 ([M+1]⁺)

The maximal absorption wavelength of an absorption spectrum of the compound (G-12) in a diluted solution of ethyl acetate was 668 nm.

### Synthesis Example 13: Synthesis of Compound (G-13)

A compound (G-13) was obtained in the same manner as in Synthesis Example 1, except that 388 mg of zinc iodide was changed to 112 mg of copper acetate (anhydrous) and benzonitrile as the solvent was changed to ethylene glycol. MALDI-TOF MS: 2128 ([M+1]⁺)

The maximal absorption wavelength of an absorption spectrum of the compound (G-13) in a diluted solution of ethyl acetate was 639 nm.

### Synthesis Example 14: Synthesis of Compound (G-14)

A compound (G-14) was obtained in the same manner as in Synthesis Example 1, except that 388 mg of zinc iodide was changed to 58 mg of vanadium trichloride and benzonitrile as the solvent was changed to diethylene glycol monobutyl ether. MALDI-TOF MS: 2132 ([M+1]⁺)

The maximal absorption wavelength of an absorption spectrum of the compound (G-14) in a diluted solution of ethyl acetate was 679 nm.

### Synthesis Example 15: Synthesis of Compound (G-16)

A compound (G-16) was obtained in the same manner as in Synthesis Example 1, except that 2.11 g of 3,6-difluoro-4,5-bis[4-(allyloxycarbonyl)phenoxy]phthalonitrile was changed to 2.45 g of 3,6-difluoro-4,5-bis[4-((2,2,2,-trifluoroethyl)oxycarbonyl)phenoxy)phthalonitrile. MALDI-TOF MS: 2465 ([M+1]⁺)

The maximal absorption wavelength of an absorption spectrum of the compound (G-16) in a diluted solution of ethyl acetate was 664 nm.

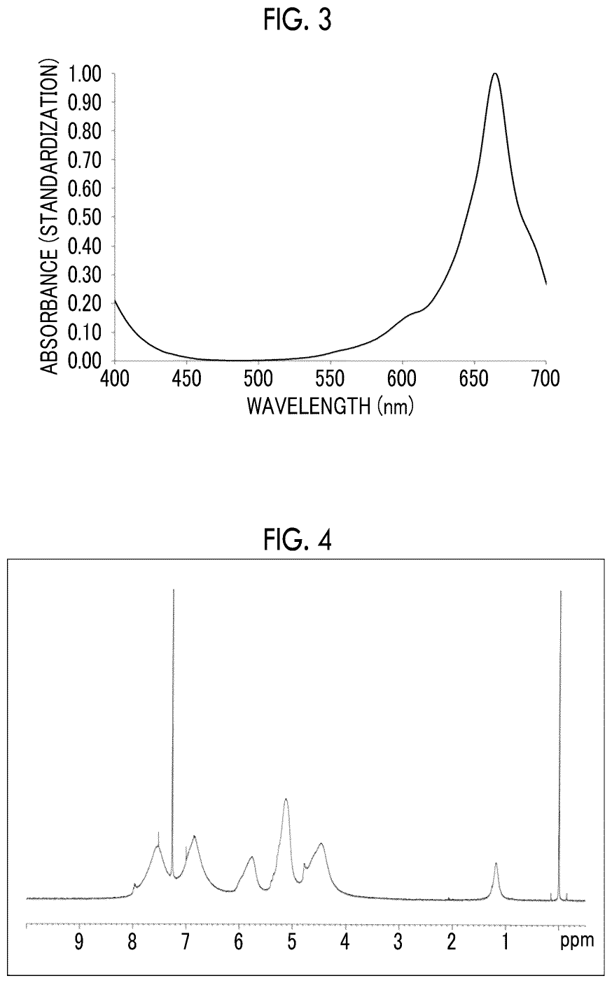

FIG. 3 shows the absorption spectrum of the compound (G-16) in the diluted solution of ethyl acetate.

### <Production of Inkjet Ink (1)>

7.5 g of the compound (G-1), 7.04 g of sodium dioctylsulfosuccinate, 4.22 g of tri(m-tolyl)phosphine oxide, 5.63 g of tri(tert-octyl)phosphine oxide, and 50 mL of ethyl acetate were mixed and dissolved at 70° C. 500 ml of deionized water was added to the solution while stirring with a magnetic stirrer, so as to prepare an oil-in-water type coarse dispersion. Next, the coarse particle dispersion was passed through a microfluidizer (manufactured by MICROFLUIDEX Inc.) five times at a pressure of 60 MPa to obtain fine particles. Furthermore, the solvent was removed from the obtained emulsified product by a rotary evaporator until the odor of ethyl acetate disappeared. 140 g of diethylene glycol, 50 g of glycerin, 7 g of SURFYNOL 465 (Air Products & Chemicals Inc.), and 900 ml of deionized water were added to the fine emulsion of the compound (G-1) thus obtained to produce an ink.

### <Production of Ink Jet Inks (2) to (14)>

Inkjet inks (2) to (14) were obtained in the same manner as in the production of the inkjet ink (1), except that any of the compounds (G-2) to (G-14) was used instead of the compound (G-1).

### <Preparation of Inkjet Inks (A) and (B)>

Inkjet inks (A) and (B) were obtained in the same manner as in the production of the inkjet ink (1), except that the following comparative compound (A) or the following comparative compound (B) was used instead of the compound (G-1).

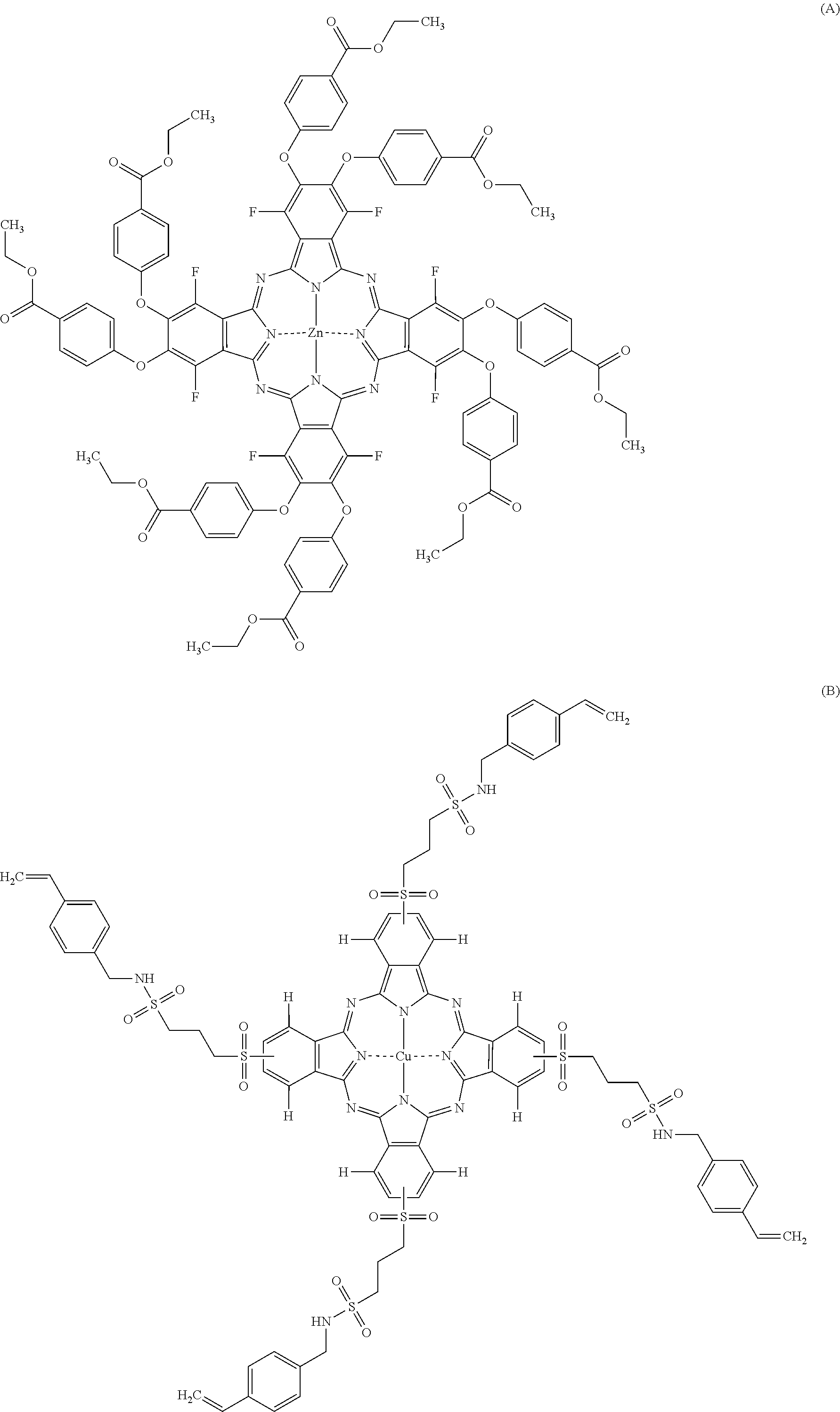

### (Preparation and Evaluation of Printed Sample)

Each of the inkjet inks (1) to (14). (A), and (B) was used to print a solid image having a reflection density of 1.0 and a solid image having the maximum color density on art paper using an inkjet printer (manufactured by FUJIFILM Corporation, trade name: Material printer DMP-2850), thereby obtaining a printed sample.

Using the obtained printed sample, the following evaluations of light fastness, measurements of hue angle, visual color check, and measurements of maximum color density were performed. The results are shown in Table 1.

### <Evaluation of Light Fastness>

The obtained printed sample was irradiated with xenon light (85,000 lux) for 7 days using a weather-meter (manufactured by Atlas, Ci65), and a reflection density of the solid image, which was 1.0 as the reflection density before the irradiation with xenon light, was measured using a reflection densitometer (manufactured by X-Rite, trade name: X-Rite ilPro) after the irradiation with xenon light. In addition, since the reflection density of the solid image before the irradiation with xenon light was 1.0, a compound residual rate (%) before and after the irradiation with xenon light was calculated from the following expression. As the compound residual rate (%) is higher, the light fastness of the fluorinated phthalocyanine compound contained in the printed sample is more excellent.

$$Compound residual rate ( \% ) = ( Reflection density of solid image after irradiation with xenon light ) / ( Reflection density of solid image before irradiation with xenon light = 1. ) \times 100$$

### <Calculation of Hue Angle>

A hue angle in the solid image of the obtained printed sample, having a reflection density of 1.0, was calculated as follows. First, with the solid image of the printed sample, having a reflection density of 1.0, using a reflection densitometer (manufactured by X-Rite, trade name: X-Rite ilPro), a color value L*a*b* was measured at an angle of view of 2 degrees and under the condition of a C light source. The obtained a* and b* were substituted into an expression: hue angle (h°)=tan-1 (a*/b*), and the hue angle in the solid image of the printed sample, having a reflection density of 1.0, was calculated.

### <Color>

The color of the obtained solid image of the printed sample, having a reflection density of 1.0, was visually confirmed.

### <Maximum Color Density>

The reflection density of the obtained solid image of the printed sample, having a maximum color density, was measured at an angle of view of 2 degrees and under the condition of a C light source, using a reflection densitometer (manufactured by X-Rite, trade name: X-Rite ilPro).

TABLE 1

| | | Phthalocyanine | | Evaluation result and measured value | | | |
|---|---|---|---|---|---|---|---|
| | Ink | compound | | Light | Hue angle | Visual | Maximum |
| | No. | No. | λ max | fastness | [°] | color | color density |
| Example 1 | (1) | (G-1) | 664 | 81% | 179 | Green | 2.39 |
| Example 2 | (2) | (G-2) | 664 | 82% | 180 | Green | 2.37 |
| Example 3 | (3) | (G-3) | 664 | 82% | 181 | Green | 2.36 |
| Example 4 | (4) | (G-4) | 664 | 82% | 181 | Green | 2.33 |
| Example 5 | (5) | (G-5) | 664 | 84% | 180 | Green | 2.38 |
| Example 6 | (6) | (G-6) | 664 | 80% | 180 | Green | 2.4 |
| Example 7 | (7) | (G-7) | 664 | 81% | 179 | Green | 2.41 |
| Example 8 | (8) | (G-8) | 665 | 83% | 180 | Green | 2.33 |
| Example 9 | (9) | (G-9) | 663 | 84% | 182 | Green | 2.39 |
| Example 10 | (10) | (G-10) | 664 | 83% | 181 | Green | 2.36 |
| Example 11 | (11) | (G-11) | 664 | 81% | 179 | Green | 2.38 |
| Example 12 | (12) | (G-12) | 668 | 81% | 180 | Green | 2.48 |
| Example 13 | (13) | (G-13) | 639 | 88% | 201 | Green | 2.28 |
| Example 14 | (14) | (G-14) | 679 | 76% | 171 | Green | 1.89 |
| Comparative | (A) | (A) | 664 | 64% | 189 | Green | 2.28 |
| Example 1 | | | | | | | |
| Comparative | (B) | (B) | 628 | 89% | 238 | Cyan | 1.89 |
| Example 2 | | | | | | | |

As is clear from Table 1, it was found that the fluorinated phthalocyanine compound according to the present disclosure (that is, the fluorinated phthalocyanine compound represented by Formula (1)) had a hue angle in a range of 150° to 210°, exhibited green color, and also had excellent light fastness.

In addition, it was found that, by using the fluorinated phthalocyanine compound according to the present disclosure (that is, the fluorinated phthalocyanine compound represented by Formula (1)), an image having a high maximum color density was obtained.

It was found that the comparative compound (A) used in Comparative Example 1 exhibited green color, but had deteriorated light fastness compared to Examples.

The comparative compound (B) used in Comparative Example 2 had excellent light fastness, but the hue angle was outside the range of 150° to 210°, and the color was cyan, not green.

The disclosure of JP2021-188790 filed on Nov. 19, 2021 is incorporated in the present specification by reference. All documents, patent applications, and technical standards described in the present specification are herein incorporated by reference to the same extent that each individual document, patent application, or technical standard was specifically and individually indicated to be incorporated by reference.

What is claimed is:

1. A fluorinated phthalocyanine compound represented by Formula (1): 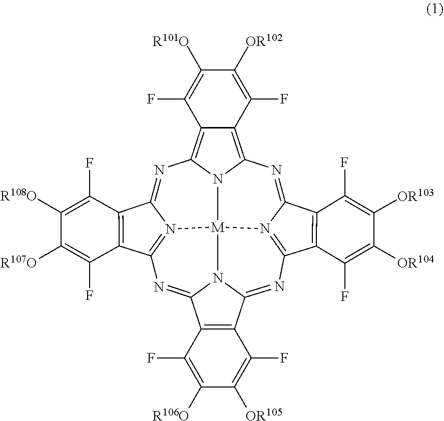
in Formula (1), M represents a metal atom or an oxide of a metal atom, and R¹⁰¹, R¹⁰², R¹⁰³, R¹⁰⁴, R¹⁰⁵, R¹⁰⁶, R¹⁰⁷, and R¹⁰⁸ each independently represent a substituted or unsubstituted alkyl group, a substituted or unsubstituted aryl group, or a substituted or unsubstituted heterocyclic group, provided that at least one of R¹⁰¹, R¹⁰², R¹⁰³, R¹⁰⁴, R¹⁰⁵, R¹⁰⁶, R¹⁰⁷, or R¹⁰⁸ is a group represented by Formula (2): 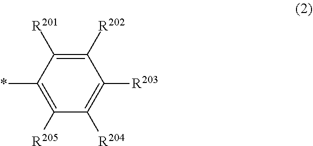
in Formula (2), R²⁰¹, R²⁰², R²⁰³, R²⁰⁴, and R²⁰⁵ each independently represent a hydrogen atom or a monovalent substituent, provided that at least one of R²⁰¹, R²⁰², R²⁰³, R²⁰⁴, or R²⁰⁵ is a group represented by any one selected from the group consisting of Formula (3), Formula (4), Formula (5), Formula (6), and Formula (7): 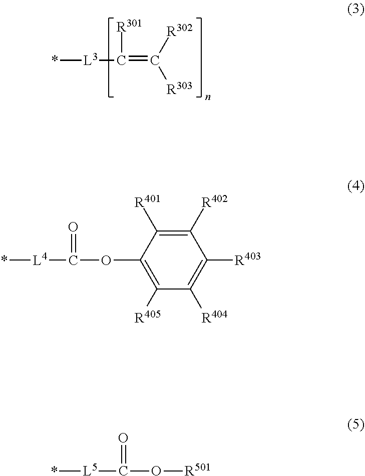
*-L⁶-OH (6)
*-L⁷-NH₂ (7)
in Formula (3), R³⁰¹, R³⁰², and R³⁰³ each independently represent a hydrogen atom or a monovalent substituent, L³ represents a single bond, a divalent linking group, or a trivalent linking group, and n represents 1 or 2;
in Formula (4), R⁴⁰¹, R⁴⁰², R⁴⁰³, R⁴⁰⁴, and R⁴⁰⁵ each independently represent a hydrogen atom or a monovalent substituent, and L⁴ represents a single bond or a divalent linking group;
in Formula (5), L⁵ represents a single bond or a divalent linking group, R⁵⁰¹ represents a hydrogen atom, a fluoroalkyl group, —CR⁵⁰²R⁵⁰³R⁵⁰⁴, or —CHR⁵⁰⁵OR⁵⁰⁶, R⁵⁰², R⁵⁰³, R⁵⁰⁴, R⁵⁰⁵, and R⁵⁰⁶ each independently represent a substituted or unsubstituted alkyl group, and R⁵⁰⁵ and R⁵⁰⁶ may be bonded to each other to form a ring;
in Formula (6), L⁶ represents a single bond or a divalent linking group; and
in Formula (7), L⁷ represents a single bond or a divalent linking group.

2. The fluorinated phthalocyanine compound according to claim 1,
wherein each of R¹⁰¹, R¹⁰², R¹⁰³, R¹⁰⁴, R¹⁰⁵, R¹⁰⁶, R¹⁰⁷, and R¹⁰⁸ is the group represented by Formula (2).

3. The fluorinated phthalocyanine compound according to claim 1,
wherein, in Formula (2), any one of R²⁰², R²⁰³, or R²⁰⁴ is a group represented by any one selected from the group consisting of Formula (3), Formula (4), Formula (5), Formula (6), and Formula (7), and the remaining two of R²⁰², R²⁰³, and R²⁰⁴, R²⁰¹, and R²⁰⁵ are all hydrogen atoms.

4. The fluorinated phthalocyanine compound according to claim 3,
wherein, in Formula (2), R²⁰³ is a group represented by any one selected from the group consisting of Formula (3), Formula (4), Formula (5), Formula (6), and Formula (7), and R²⁰¹, R²⁰², R²⁰⁴, and R²⁰⁵ are all hydrogen atoms.

5. The fluorinated phthalocyanine compound according to claim 1,
wherein M is copper, zinc, or oxyvanadium.

6. The fluorinated phthalocyanine compound according to claim 5,
wherein M is copper or zinc.

7. The fluorinated phthalocyanine compound according to claim 6,
wherein M is zinc.

8. A coloring composition comprising:
the fluorinated phthalocyanine compound according to claim 1.

9. An inkjet ink comprising:
the fluorinated phthalocyanine compound according to claim 1.
